(19) United States
(12) Patent Application Publication
(10) Pub. No.: US 2026/0249044 A1
(43) Pub. Date: Aug. 27, 2026

(54) INTEGRATED ELECTRODE LUMENS WITH ACCESS THROUGH DISCRETE BRAID

(71) Applicant: Boston Scientific Scimed, Inc., Maple Grove, MN (US)

(72) Inventors: Natalie Nicole Cichon, Etobicoke (CA); Miles Montgomery, East Gwillimbury (CA); Melaine Dhaliwal, Brampton (CA)

(21) Appl. No.: 19/550,005

(22) Filed: Feb. 25, 2026

Related U.S. Application Data
(60) Provisional application No. 63/764,321, filed on Feb. 27, 2025.

(51) Int. Cl. A61M 25/00; A61M 25/01; A61M 25/06

(52) U.S. Cl. CPC A61M 25/005; A61M 25/0012; A61M 25/0045; A61M 2025/0166; A61M 2025/0681; A61M 2205/0238

(57) ABSTRACT

A medical device includes an elongated tubular member having a proximal portion with a proximal end and a distal portion with a distal end. The elongated tubular member includes an inner sleeve. The inner sleeve has an outer surface extending between an inner sleeve proximal end and an inner sleeve distal end. At least one guide member is located along a portion of the outer surface of the inner sleeve. A braided layer surrounds the at least one guide member and the inner sleeve. The braided layer includes a braid pattern extending along the at least one guide member having a consistent pick size along a first section, a consistent pick size along a second section, and a first larger pick between the first section and the second section. A layer of polymeric material encases the braided layer and the at least one guide member.

### CROSS REFERENCE TO RELATED APPLICATIONS

This application claims priority to U.S. Provisional Patent Application No. 63/764,321 entitled “INTEGRATED ELECTRODE LUMENS WITH ACCESS THROUGH DISCRETE BRAID,” filed February 27, 2025, which is hereby incorporated by reference in its entirety.

### TECHNICAL FIELD

The present disclosure relates to medical systems and methods for electrically coupling electrodes. More specifically, the present disclosure relates to medical devices including guide lumens for conductors.

### BACKGROUND

Medical devices such as catheters, guidewires, sheath, and dilators often include electrodes for various reasons. For example, electrodes can be used for ablation, sensing, and for use with electroanatomical mapping (EAM). Commonly, medical devices include support layers such as braids that extend along a length of the medical device. Conductors which couple the electrodes to a control system must transition from underneath the support layer to couple with the electrodes at the outer surface of the medical device. This requires a break-out process that requires tedious manual labor and training time. This break-out process also increases risks of leakage and introduces weak points that are susceptible to kinking.

### SUMMARY

Example 1 is a medical device. The medical device includes an elongated tubular member having a proximal portion including a proximal end and a distal portion including a distal end. The elongated tubular member includes an inner sleeve having an outer surface extending between an inner sleeve proximal end and an inner sleeve distal end. At least one guide member is located along a portion of the outer surface of the inner sleeve. A braided layer surrounds the at least one guide member and the inner sleeve. The braided layer includes a braid pattern extending along the at least one guide member having a consistent pick size along a first section, a consistent pick size along a second section, and a first larger pick between the first section and the second section.

Example 2 is the medical device of Example 1, wherein the braid pattern includes a consistent pick size along a third section, and a second larger pick between the second section and the third section.

Example 3 is the medical device of Example 2, wherein the braid pattern includes a consistent pick size along a fourth section, and a third larger pick between the third section and the fourth section.

Example 4 is the medical device of Example 3, wherein the braid pattern includes a consistent pick size along a fifth section, and a fourth larger pick between the fourth section and the fifth section.

Example 5 is the medical device of Example 4, wherein the first larger pick, the second larger pick, the third larger pick, and the fourth larger pick are the same size.

Example 6 is the medical device of any of Examples 1 to 5, wherein a distal end of the at least one guide member is adjacent the inner sleeve distal end.

Example 7 is the medical device of any of Examples 1 to 6, wherein the at least one guide member includes a first guide member and a second guide member.

Example 8 is the medical device of Example 7, wherein the first guide member is located opposite the second guide member.

Example 9 is the medical device of any of Examples 1 to 8, further comprising at least one conductor extending through the at least one guide member.

Example 10 is the medical device of any of Examples 1 to 9, further comprising a first electrode positioned over the first larger pick.

Example 11 is the medical device of Example 10, wherein the first electrode is electrically connected to the at least one conductor.

Example 12 is the medical device of any of Examples 1 to 11, wherein the braided layer is formed of stainless-steel wire, nickel-titanium wire, metallic wire, polymeric wire, or fiber wire.

Example 13 is the medical device of any of Examples 1 to 12, further comprising a layer of polymeric material encasing the braided layer and the at least one guide member.

Example 14 is the medical device of any of Examples 1 to 13, further comprising a connector at the proximal end of the elongated tubular member.

Example 15 is the medical device of any of Examples 1 to 14, wherein the elongated tubular member includes a lumen extending between the proximal end and the distal end.

Example 16 is a medical device. The medical device includes an elongated tubular member having a proximal portion with a proximal end and a distal portion with a distal end. The elongated tubular member includes an inner sleeve. The inner sleeve has an outer surface extending between an inner sleeve proximal end and an inner sleeve distal end. At least one guide member is located along a portion of the outer surface of the inner sleeve. A braided layer surrounds the at least one guide member and the inner sleeve. The braided layer includes a braid pattern extending along the at least one guide member having a consistent pick size along a first section, a consistent pick size along a second section, and a first larger pick between the first section and the second section. A layer of polymeric material encases the braided layer and the at least one guide member.

Example 17 is the medical device of Example 16, wherein the braid pattern includes a consistent pick size along a third section, and a second larger pick between the second section and the third section.

Example 18 is the medical device of Example 17, further comprising a first channel extending through the first larger pick and a second channel extending through the second larger pick.

Example 19 is the medical device of Example 17, wherein the first larger pick and the second larger pick are the same size, or the first larger pick and the second larger pick are different sizes.

Example 20 is the medical device of Example 16, wherein a distal end of the at least one guide member is adjacent the inner sleeve distal end.

Example 21 is the medical device of Example 16, wherein the at least one guide member includes a first guide member and a second guide member.

Example 22 is the medical device of Example 21, wherein the first guide member is located opposite the second guide member.

Example 23 is the medical device of Example 16, further comprising at least one conductor extending through the at least one guide member.

Example 24 is the medical device of Example 16, further comprising a first electrode positioned over the first larger pick.

Example 25 is the medical device of Example 24, wherein the first electrode is electrically connected to at least one conductor extending through the at least one guide member.

Example 26 the medical device of Example 25, wherein the at least one conductor passes through a first channel extending through the first larger pick.

Example 27 is the medical device of Example 26, further comprising a connector at the proximal end of the elongated tubular member, the at least one conductor being electrically coupled to the connector.

Example 28 is a medical device. The medical device includes an elongated tubular member having a proximal portion with a proximal end and a distal portion with a distal end. The elongated tubular member includes an inner sleeve having an outer surface extending between an inner sleeve proximal end and an inner sleeve distal end. At least one guide member is located along a portion of the outer surface of the inner sleeve. The at least one guide member has a first lateral opening. A braided layer surrounds the at least one guide member and the inner sleeve. The braided layer includes a braid pattern extending along the at least one guide member. The braid patterns has a consistent pick size along a first section, a consistent pick size along a second section, and a first larger pick between the first section and the second section. The first larger pick surrounds the first lateral opening.

Example 29 is the medical device of Example 28, further comprising a first electrode positioned over the first larger pick.

Example 30 is the medical device of Example 29, further comprising at least one conductor extending through the at least one guide member and the first lateral opening, the at least one conductor being electrically coupled to the first electrode.

Example 31 is a method of making a medical device. The method includes placing at least one guide member on an outer surface of an inner sleeve. The method includes braiding a support layer around the at least one guide member and the inner sleeve. The support layer includes a braid pattern extending along the at least one guide member having a consistent pick size along a first section, a consistent pick size along a second section, and a first larger pick between the first section and the second section.

Example 32 is the method of making a medical device of Example 31, wherein the first larger pick is formed by braiding around a mandrel positioned transverse to a longitudinal axis of the support layer.

Example 33 is the method of making a medical device of Example 31, further comprising surrounding the at least one guide member, the inner sleeve, and the support layer with a polymeric material.

Example 34 is the method of making a medical device of Example 33, further comprising forming a channel through the polymeric material, the channel passing through the first larger pick and into the at least one guide member.

Example 35 is the method of making a medical device of Example 34, further comprising extending a conductor through the channel.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a schematic overview of a system for applying a braid to a medical device, in accordance with embodiments of the disclosure.

FIG. 2 is an illustration of a portion of a braid having a radially uniform braid density with consistent pick sizes, in accordance with embodiments of the disclosure.

FIG. 3 is a perspective view of using a pick modifying device to increase the size of a pick at a desired location, in accordance with embodiments of the disclosure.

FIG. 4 is a perspective, partial cross-sectional view of a medical device during assembly, in accordance with embodiments of the disclosure.

FIG. 5 is a partial view of a portion of a braided layer having a larger pick overlying a guide member, in accordance with embodiments of the disclosure.

FIG. 6 is a partial view of a portion of a braided layer having a plurality of larger picks overlying a guide member, in accordance with embodiments of the disclosure.

FIG. 7 is a cross-sectional view of a medical device, in accordance with embodiments of the disclosure.

FIG. 8 is a cross-sectional view of the medical device of FIG. 7 along line 8-8, in accordance with embodiments of the disclosure.

FIG. 9 is a cross-sectional view of a medical device including multiple guide members, in accordance with embodiments of the disclosure.

FIGS. 10A – 10E illustrate various cross-sections for filaments forming a braided layer, in accordance with embodiments of the disclosure.

FIG. 11 is a flowchart illustrating a method of making a medical device, in accordance with embodiments of the disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

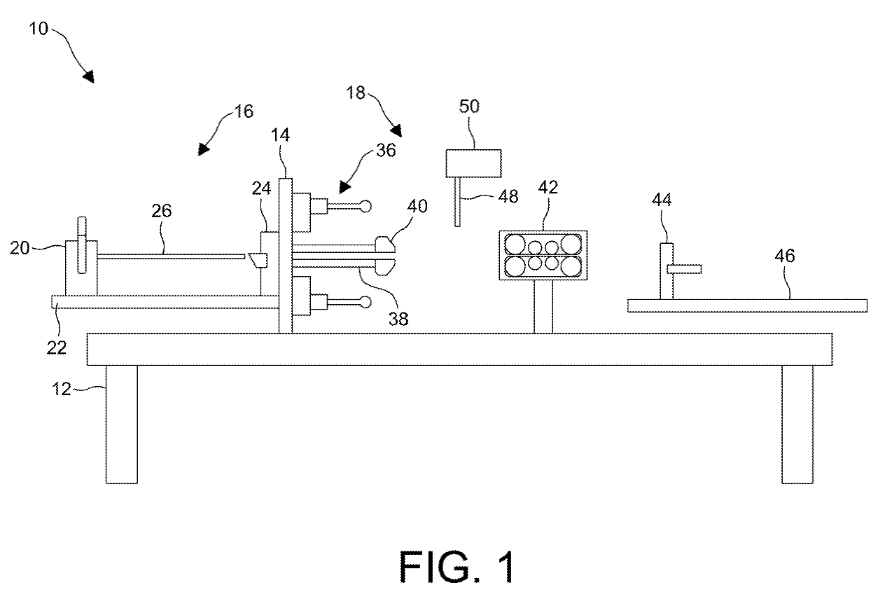

FIG. 1 is a schematic overview of a system 10 for applying a braid to a medical device, in accordance with embodiments of the disclosure. The system includes a support 12 configured to securely support the components of the system 10. The support 12 can take the form of a table, a series of rails joined together, or other structure capable of supporting the components of the system 10. The system 10 includes a wall 14 that separates an infeed portion 16 from an outfeed portion 18. As a tubular member is braided, it moves from the infeed portion 16 towards the outfeed portion 18.

The infeed portion 16 includes an infeed clamp 20 mounted on an infeed rail 22 and an infeed guide 24 attached to the wall 14. The infeed clamp 20 is configured to move along the infeed rail 22 towards the infeed guide 24 during the braiding process. The infeed clamp 20 is configured to prevent rotation of a workpiece 26, and one or more guide member during the braiding process. A workpiece 26 includes a tubular member on which a braid is to be applied as well as a support piece. For example, a workpiece 26 can include a stainless-steel mandrel, a tubular member, a polymeric monofilament, or a tubular member mounted on a stainless-steel mandrel. The infeed guide 24 is configured to maintain the one or more guide member in a desired axial location along the workpiece 26. The one or more guide member can include a single guide member or a plurality of guide members. The one or more guide member is used as a channel to guide one or more conductor or other embedded metallic or filament wire as discussed below. As used herein, the term “conductor” includes metallic wires, stranded conductors, conductive filaments, or other electrically conductive elements suitable for transmitting electrical signals or energy.

The outfeed portion 18 of the system 10 includes a braiding machine 36 having a spindle 38 through which the workpiece 26 passes during application of the braid. The spindle 38 may include magnets to aid in alignment of one or more guiding member along the workpiece 26. Spindle 38 is configured to translate axially toward the infeed portion 16 or toward the outfeed portion 18. As known in the art and not described herein, the braiding machine 36 includes one or more bobbins for holding a filament forming the braid, carriers to hold and guide the bobbins through the braiding process, and horn gears to drive the carriers in a specific pattern to create the desired braid. Located at a distal end of the spindle 38 is a nose cone 40 over which filaments slide during the braiding process. A belt driven conveyor 42 or linear actuator moves the workpiece 26 towards an outfeed clamp 44. The outfeed clamp 44 secures a distal end of the workpiece 26 and is translatable along an outfeed rail 46. As the conveyor 42 drives the workpiece 26, the outfeed clamp 44 holding the workpiece 26 moves along the outfeed rail 46 away from the wall 14.

While FIG. 1 illustrates one example of a discrete horizontal braiding system 10 including infeed clamp 20 and outfeed clamp 40 and associated rails 22, 46, it is understood that the illustrated system 10 is exemplary only. In other embodiments, the braided layer is formed using alternative braiding configurations, including reel-to-reel braiding systems, vertical braiders, or other continuous or discontinuous braiding machines. One or more of the illustrated clamps, rails, or support components may be omitted, modified, or replaced depending on the selected braiding system.

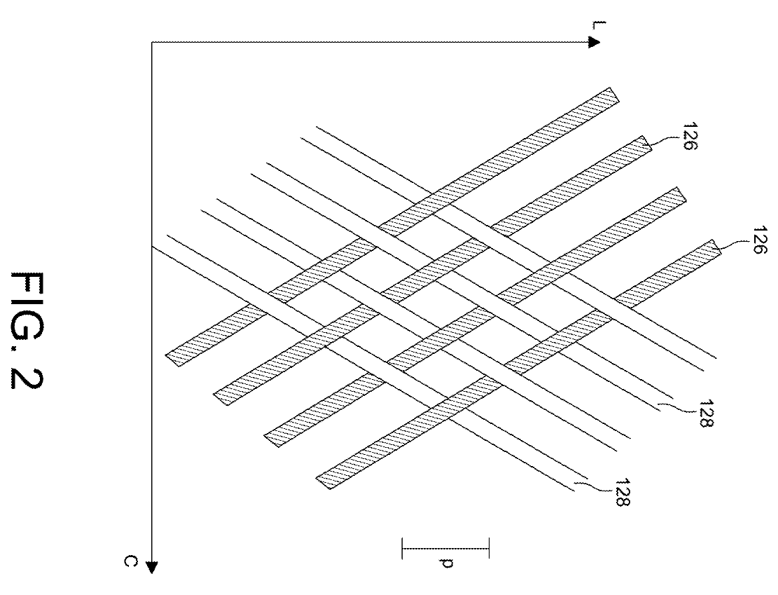

The system 10 is configured to form a braid having a consistent pick size, also know as a uniform braid density, or a braid having a variable braid density. The system 10 is also configured to provide picks of increased size at discrete locations. FIG. 2 is an illustration of a braid having consistent pick size, in accordance with the disclosure. One example of a braid having a uniform braid density is illustrated in FIG. 2. As shown, the braid is formed of a plurality of filaments 126, 128. The plurality of filaments 126, 128 are shown in different colors for ease of presentation. In some embodiments, the plurality of filaments 126, 128 may be formed of a single material. In some embodiments, the plurality of filaments 126, 128 may be formed of a plurality of materials. In some embodiments, the plurality of filaments 126,128 may have the same size and cross-sectional shape. In some embodiments, the plurality of filaments 126,128 may have different sizes and cross-sectional shapes.

The plurality of filaments 126, 128 are braided together to form a uniform braid density around the circumference and along the length of a medical device. In FIG. 2, the braided portion is illustrated as flat. The circumference of the medical device is illustrated in the C direction, while the longitudinal axis of the medical device is illustrated in the L direction. As can be seen in FIG. 2, the picks P of the braided section are uniform and consistent in size. As used herein, the term “pick” refers to a crossing region formed by intersecting filaments 126, 128, corresponding to a repeating unit of the braid pattern along a longitudinal axis of the medical device. In some embodiments, pick size is defined by the axial spacing between adjacent filament crossings and/or the size of an opening formed between intersecting filaments 126, 128.

While the braid in FIG. 2 is illustrated as having single filaments braided together, it is understood that other arrangements of filaments and different braid patterns can be used. For example, the braid can be formed of groups of two filaments that are braided together, where two filaments are adjacent one another, then side by side alternately pass under two filaments, then over two filaments, and so on. Although the illustrated embodiments depict a 1‑over‑1 braid pattern with substantially uniform braid density, other braid patterns may be used, including but not limited to 2‑over‑1, 2‑over‑2, or multi‑strand braid configurations.

In some embodiments, the filaments 126, 128 forming the braided layer includes metallic wires, polymeric monofilaments, composite filaments, or combinations thereof, including polymer-core filaments such as PTFE-core or acetal-core filaments. Additionally, the braid can include additional filaments having different properties than filaments 126, 128. For example, the braid can include one or more radiopaque filaments. In some embodiments, the braid pattern includes one or more larger picks disposed at selected axial and circumferential locations, depending on the location of underlying guide members 224 or other structures. Further, the braided layer may include regions of variable braid density, with the larger picks representing localized spreading of the braid at desired locations rather than a global change in braid density. The illustrated embodiments and braid configurations are provided by way of example only, and the disclosed features may be combined, rearranged, or omitted without departing from the scope of the disclosure.

The system 10 includes a pick modifying device 48 to modify the size of a single pick at a desired axial and longitudinal location. The pick modifying device 48 is configured to increase the size of a single pick during the braiding process. The pick modifying device 48 can be associated with an actuator 50, such as a solenoid or motor, to control engagement of the pick modifying device 48. A processor (not shown) can be programed to control the actuator 50 to engage the pick modifying device 48 at one or more locations during the braiding process. In some embodiments, the pick modifying device 48 is manually controlled.

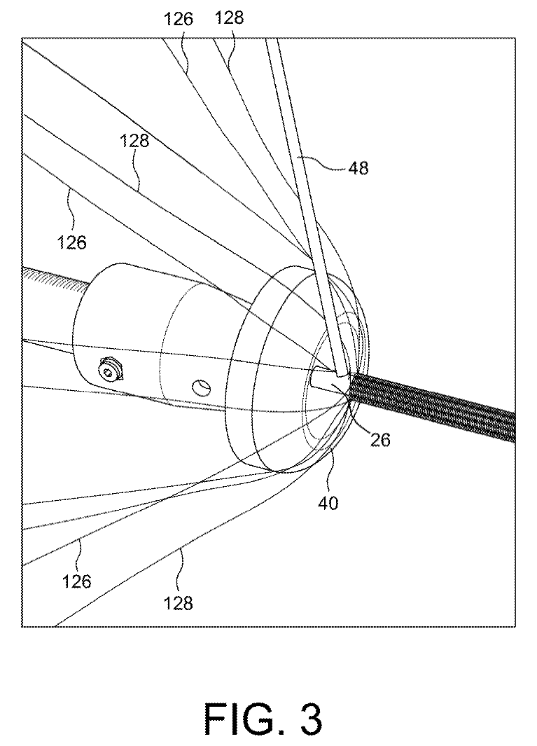

The pick modifying device 48 includes a mandrel having a circular, oval, rectangular, or polygonal cross-section. As illustrated in FIG. 3, to create a larger pick within the braid at a desired location, the pick modifying device 48 is inserted within the filaments 126, 128 distal of the nose cone 40 before the filaments 126, 128 are braided together and lay flat against the workpiece 26. The filaments 126, 128 are then braided around the pick modifying device 48 leaving a larger pick in the braid when the pick modifying device 48 is removed. In other embodiments, the system 10 is configured to produce a braid having larger picks at desired locations, for example by incorporating a cartesian braider.

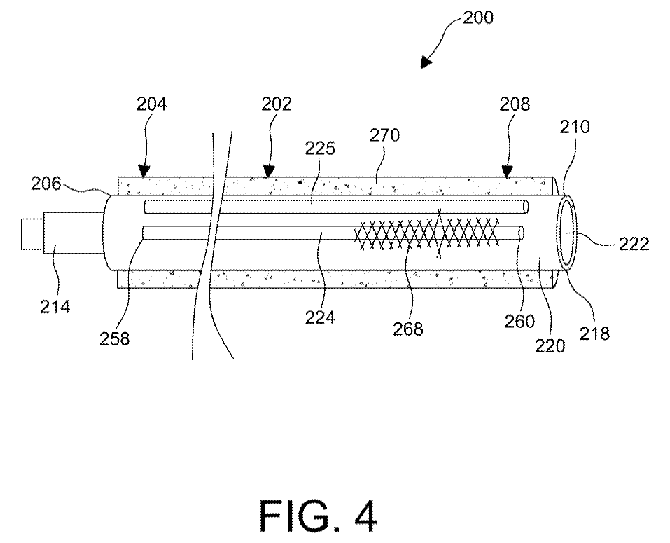

FIG. 4 is a perspective, partial cross-sectional view of a medical device 200 during assembly, in accordance with embodiments of the disclosure. The medical device 200 can take the form of a sheath, catheter, dilator, perforation device, guidewire, or any other elongate medical device capable of being used within a patient. The medical device 200 includes an elongated tubular member 202 having a proximal portion 204 including a proximal end 206. The elongated tubular member 202 includes a distal portion 208 having a distal end 210. In some embodiments, the elongated tubular member 202 has a constant diameter from the proximal end 206 to the distal end 210. In some embodiments, the elongated tubular member 202 includes a tapered distal portion that reduces in diameter towards the distal end 210. The proximal end 206 of the elongated tubular member 202 includes a connector 214 to electrically couple one or more electrodes (not shown) to a control system, for example an EAM system or an RF generator.

The elongated tubular member 202 is formed of multiple layers. The elongated tubular member 202 includes an inner sleeve 218 having an outer surface 220. The inner sleeve 218 includes a lumen 222 that extends between the proximal end 206 and the distal end 210. The inner sleeve 218 is formed of a suitable polymeric material. A braided layer 268 and a layer of material 270 also form part of the elongated tubular member 202 as discussed further below.

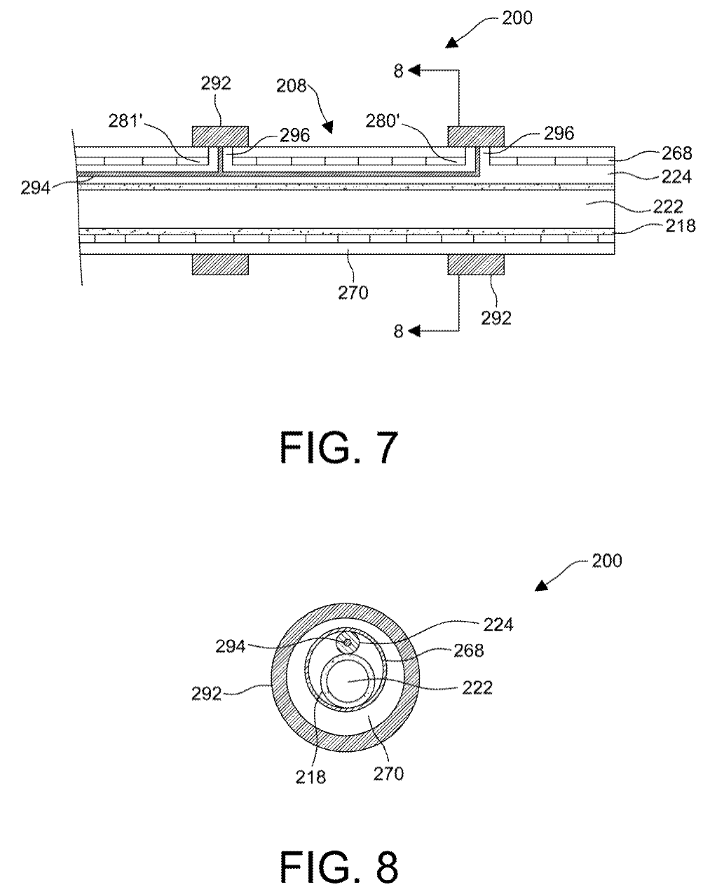

Located along the outer surface 220 of the inner sleeve 218 is at least one guide member 224. The at least one guide member 224 is an elongated tubular member arranged substantially parallel to a longitudinal axis of the elongated tubular member 202. The at least one guide member 224 is formed at least partially of a polymeric material, for example Pebax, PTFE, or a composite thereof. In some embodiments, the at least one guide member 224 includes a support layer, such as a braid or coil. The at least one guide member 224 extends from the proximal portion 204 to the distal portion 208. In some embodiments, a guide member proximal end 258 is adjacent the inner sleeve proximal end 206 and a guide member distal end 260 is adjacent the inner sleeve distal end 210. In some embodiments, the guide member proximal end 258 is spaced from the inner sleeve proximal end 206 and the guide member distal end 260 is spaced from the inner sleeve distal end 210. The at least one guide member 224 is configured to guide at least one conductor 294 (FIG. 7) from the proximal portion 204 to the distal portion 208. The at least one conductor 294 electrically couples one or more electrodes 292 (FIG. 7) located in the distal portion 208 to the connector 214, and thus the control system. While not illustrated, the at least one guide member 224 can include a plurality of guide members spaced around the inner sleeve 218. In one embodiment, the plurality of guide members are equally spaced around the inner sleeve 218. In other embodiments, the plurality of guide members are adjacent one another.

Also located along the outer surface 220 of the inner sleeve 218 is at least one pull wire lumen 225. The at least one pull wire lumen 225 is configured to provide a channel for a control wire (not shown) that allows for deflection or changing shape of the medical device 200. The control wire (not shown) is secured near the distal end 210 and extends proximally through the at least one pull wire lumen 225 past the proximal end 206 to a control mechanism (not shown), for example an actuator. A user manipulates the control mechanism (not shown) to cause tensioning of the control wire to deflect or change the shape of the medical device 200. While not illustrated, the at least one pull wire lumen 225 can include a plurality of pull wire lumens spaced around the inner sleeve 218.

The braided layer 268, only a portion of which is illustrated in FIG. 4, surrounds the at least one guide member 224, the at least one pull wire lumen 225, and the inner sleeve 218. In some embodiments, the at least one guide member 224 and the at least one pull wire lumen 225 are triaxially integrated with the braided layer 268, having portions of the braided layer 268 both underneath and over the at least one guide member 224 and the at least one pull wire lumen 225. The braided layer 268 provides support to the medical device 200. The braided layer 268 includes a material that can improve torque transmission, increase rigidity, or otherwise improve handling of the medical device 200. In some embodiments, the braided layer 268 is formed of a metal such as stainless-steel or nickel-titanium. In some embodiments, the braided layer 268 is formed of a fabric or fiber such as Kevlar. In some embodiments, the braided layer 268 is formed of polymeric wire or metallic wire other than stainless-steel or nickel-titanium. The braided layer 268 includes portions of consistent pick size along with picks having a larger size interposed therein. In embodiments having a variable braid density, the braided layer 268 includes portions of variable pick size along with picks having a larger size interposed therein. The braided layer 268 is arranged so that any picks having a larger size are disposed over the at least one guide member 224 as discussed further below.

The layer of material 270 encases the braided layer 268 and the at least one guide member 224. The layer of material 270 can include any polymer or polymeric material and can flow in spaces between the braided layer 268 and the at least one guide member 224. In some embodiments, the layer of material 270 includes Pebax or PTFE. In some embodiments, the layer of material 270 is reflowed to encase the braided layer 268 and the at least one guide member 224. The layer of material 270 provides a smooth outer surface for the medical device 200. In some embodiments, the layer of material 270 is selected to include one or more materials that increase lubricity of the medical device 200. In some embodiments, a coating (not shown) is applied over the layer of material 270 to increase lubricity of the medical device 200. In some embodiments, the layer of material 270 and the inner sleeve 218 are formed of the same polymer or polymeric material. In some embodiments, the layer of material 270 and the inner sleeve 218 are formed of different polymers or polymeric materials. In some embodiments, the at least one guide member 224 is positioned radially inward of the braided layer 268, between the inner sleeve 218 and the braided layer 268. In other embodiments, the at least one guide member 224 is triaxially integrated with the braided layer 268, for example positioned between clockwise and counter‑clockwise filaments 126, 128 (shown in FIG. 2), partially embedded within the braid structure, or otherwise integrated with the braided layer 268.

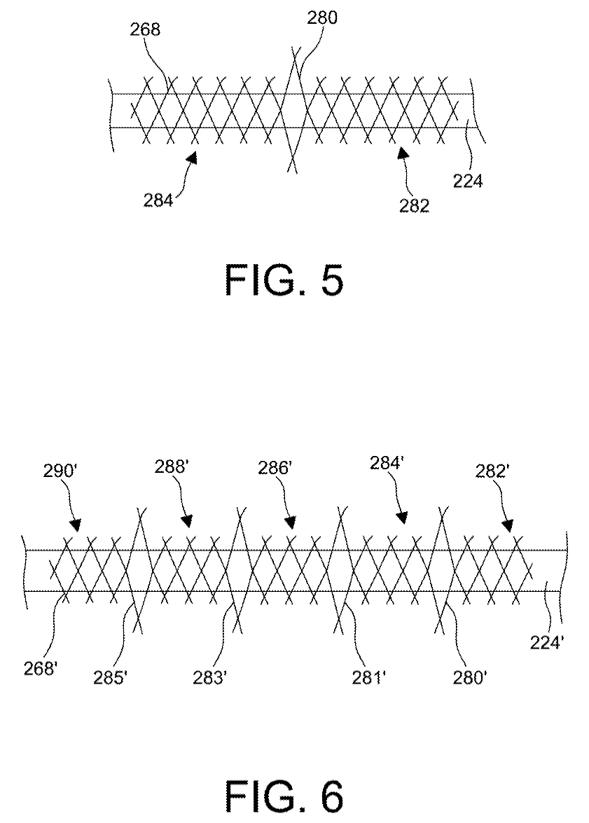

FIG. 5 is a partial view of a portion of a braided layer 268 having a larger pick 280 overlying the guide member 224, in accordance with embodiments of the disclosure. The braided layer 268 surrounds the at least one guide member 224 and includes a braid pattern that extends along the at least one guide member 224. The braid pattern includes a first section 282 having a consistent pick size and a second section 284 having a consistent pick size. A first larger pick 280 is disposed between the first section 282 and the second section 284. The first larger pick 280 is formed during the braiding process using a pick modifying device 48 as discussed above. The first larger pick 280 is arranged over the at least one guide member 224 so that a channel can be formed from the at least one guide member 224 through the interior of first larger pick 280.

FIG. 6 is a partial view of a portion of a braided layer 268’ having a plurality of larger picks overlying the at least one guide member 224’, in accordance with embodiments of the disclosure. The braided layer 268’ surrounds the at least one guide member 224’ and includes a braid pattern that extends along the at least one guide member 224’. The braid pattern includes a first section 282’ having a consistent pick size, a second section 284’ having a consistent pick size, a third section 286’ having a consistent pick size, a fourth section 288’ having a consistent pick size, and a fifth section 290’ having a consistent pick size. A first larger pick 280’ is disposed between the first section 282’ and the second section 284’. A second larger pick 281’ is disposed between the second section 284’ and the third section 286’. A third larger pick 283’ is disposed between the third section 286’ and the fourth section 288’, and a fourth larger pick 285’ is disposed between the fourth section 288’ and the fifth section 290’. The first larger pick 280’, second larger pick 281’, third larger pick 283’, and fourth larger pick 285’ are formed during the braiding process using a pick modifying device 48 as discussed above and each have the same size. In an alternate embodiment, the first larger pick 280’, second larger pick 281’, third larger pick 283’, and fourth larger pick 285’ are formed during the braiding process using a variety of pick modifying devices 48 in order to create picks having different sizes. The first larger pick 280’, second larger pick 281’, third larger pick 283’, and fourth larger pick 285’ are arranged over the at least one guide member 224 so that a channel can be formed from the at least one guide member 224 through the interior of each of the first larger pick 280’, second larger pick 281’, third larger pick 283’, and fourth larger pick 285’. While only four larger picks are illustrated in FIG. 6, the braid pattern can include any number of larger picks as desired. While FIG. 6 illustrates a plurality of larger picks arranged longitudinally along a single guide member 224, it is understood that larger picks may be positioned at any circumferential location around the elongated tubular member. Where multiple guide members 224 are present, corresponding larger picks may be circumferentially offset from one another and need not be aligned in a single axial row. In some embodiments, larger picks are formed in the braided layer 268’ overlying one or more pull wire lumens rather than, or in addition to, guide members 224 containing electrical conductors. In such embodiments, the larger picks provide localized expansion of the braided layer to accommodate pull wire lumens, facilitate access to the pull wire lumen, or reduce stress concentrations during deflection of the medical device 200. By way of example, in steerable sheaths or other deflectable medical devices, larger picks may be positioned over pull wire lumens to permit passage, anchoring, or articulation of pull wires while maintaining braid integrity and flexibility.

FIG. 7 is a cross-sectional view of a completed medical device 200 having a plurality of electrodes 292 mounted on the distal portion 208, in accordance with embodiments of the disclosure. FIG. 8A is a cross-sectional view of the medical device 200 along line 8-8 of FIG. 7. As illustrated in FIG. 7, channels 296 are formed through the layer of material 270 and into the at least one guide member 224. The channels 296 pass through the interior of the first larger pick 280’ and the second larger pick 281’. The channels 296 can be formed using a drill bit, punch, or laser prior to placement of the plurality of electrodes 292. The electrodes are placed over the channels 296, and thus the first larger pick 280’ and the second larger pick 281’. At least one conductor 294 extends from the connector 214, through the at least one guide member 224, and electrically connects to the plurality of electrodes 292 through the channels 296. In some embodiments, the medical device 200 includes a plurality of conductors such that each of the plurality of electrodes 292 are electrically connected to the connector 214 by a single conductor. While only one guide member 224 is illustrated in FIGS. 7 and 8, some embodiments may include a plurality of guide members 224, each of the plurality of guide members 224 including one or more conductors. In such a configuration, the plurality of guide members 224 can be placed around the inner sleeve 218 at various locations such that the larger picks are arranged at different circumferential locations along the medical device 200.

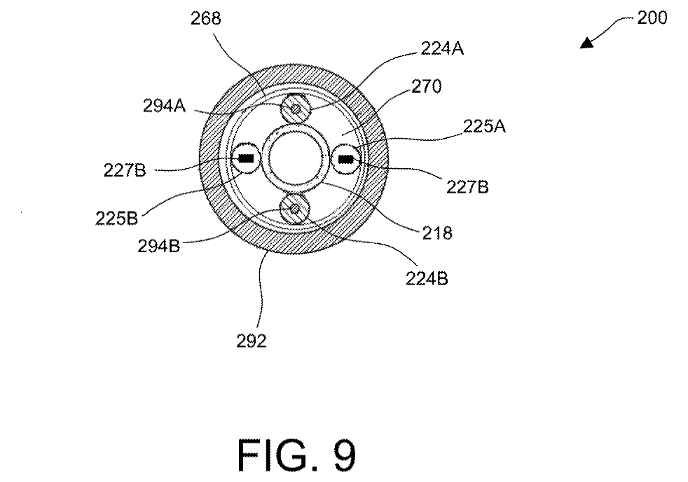

FIG. 9 is a cross-sectional view of a medical device 200 including multiple guide members, in accordance with embodiments of the disclosure. As illustrated in FIG. 9, the at least one guide member 224 includes a first guide member 224A and a second guide member 224B located on opposite sides of the inner sleeve 218. In some embodiments, the first guide member 224A and the second guide member 224B are located adjacent one another or positioned in an arrangement other than on opposite sides of the inner sleeve 218. Multiple guide members allow for connection of a plurality of electrodes 292 to a plurality of conductors 294A, 294B through channels passing through larger picks as discussed above. In this arrangement, larger picks are located on opposite sides of the medical device 200 in order to align with the first guide member 224A and the second guide member 224B. While only two guide members are illustrated in FIG. 9, it is understood that increasing the number of guide members would allow for more conductors to be provided.

The medical device 200 of FIG. 9 illustrates a plurality of pull wire lumens 225A, 225B located on opposite sides of the inner sleeve 218. The first pull wire lumen 225A provides a channel for a first control wire 227A and the second pull wire lumen 225B provides a channel for a second control wire 227B. The first control wire 227A and the second control wire 227B are positioned on opposite sides of the inner sleeve 218 to allow for deflection of the medical device 200 in two directions. While only two pull wire lumens are illustrated in FIG. 9, it is understood that increasing the number of pull wire lumens would allow for more control wires, and thus an increase in steering capabilities of the medical device 200. The guide members 224A, 224B and the pull wire lumens 225A, 225B are surrounded by the braided layer 268 that includes a plurality of larger picks positioned over the guide members 224A, 224B and embedded in a layer of material 270.

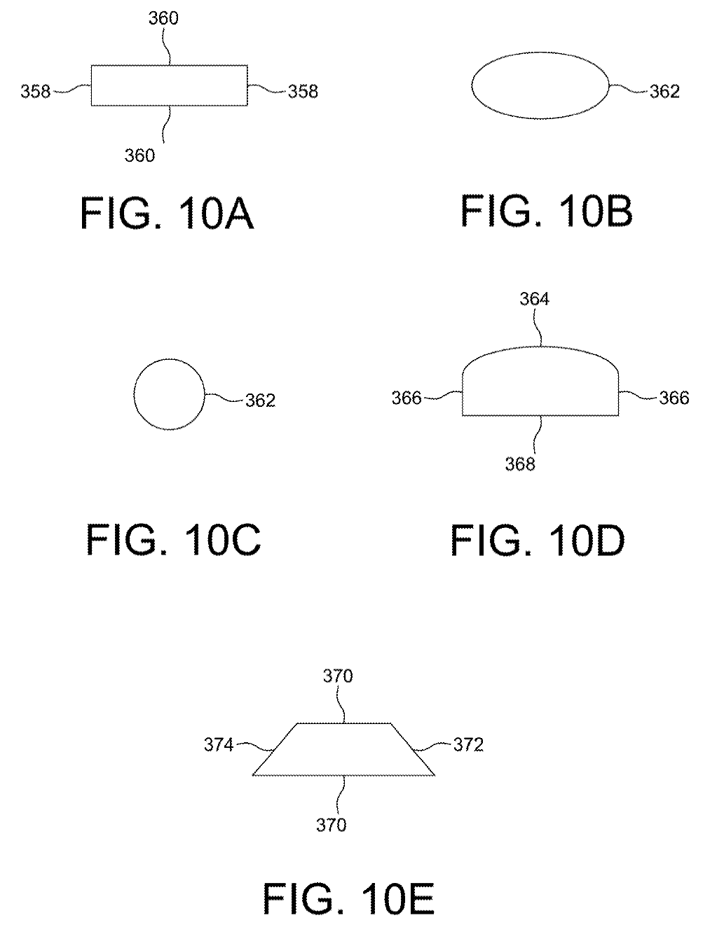

FIGS. 10A – 10E illustrate various cross-sectional arrangements for the filaments forming the braided layer 268, in accordance with the disclosure. The filaments forming the braided layer 268 can include a variety of cross-sections and can include a thickness that is less than the width. In various embodiments, the filaments have a thickness sufficient to provide structural support to the medical device 200, while maintaining substantial flexibility. FIG. 10A illustrates a filament having a rectangular cross-section. The rectangular cross-section includes a first pair of surfaces 358 that are orthogonal to a second pair of surfaces 360. FIG. 10B illustrates a filament having an oval cross-section. The oval cross-section includes a single surface 362. FIG. 10C illustrates a filament having a circular cross-section. Like the oval cross-section, the circular cross-section includes a single surface 362. FIG. 10D illustrates a filament having a dome shaped cross-section. The dome shaped cross-section includes a curved surface 364, and a first pair of parallel surfaces 366 that are orthogonal to a flat surface 368 opposite of the curved surface 364. FIG. 10E illustrates a filament having a polygonal cross-section. The polygonal cross-section includes a pair of parallel surfaces 370 that are intersected by a first angled surface 372 and a second angled surface 374.

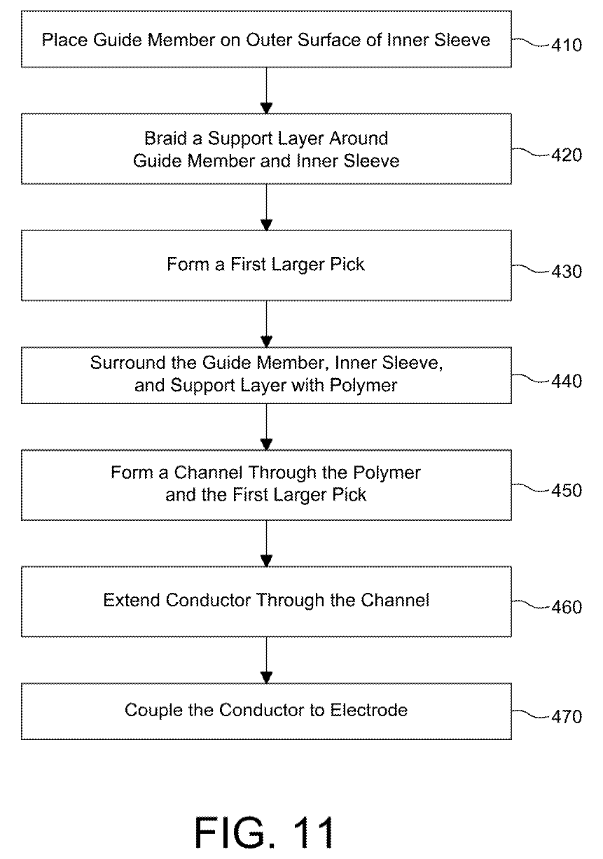

FIG. 11 illustrates a method of making a medical device, in accordance with embodiments of the disclosure. The method includes the step 410 of placing at least one guide member on an outer surface of an inner sleeve. At step 420, the method includes braiding a support layer (i.e. braided layer) around the at least one guide member and the inner sleeve. The support layer includes a braid pattern extending along the at least one guide member having a consistent pick size along a first section, a consistent pick size along a second section, and a first larger pick between the first section and the second section. At step 430, the method of making a medical device forming the first larger pick by braiding around a mandrel positioned transverse to a longitudinal axis of the support layer. Steps 420 and 430 can be repeated at various longitudinal locations along the medical device to create a plurality of larger picks separated by sections of consistent pick size. At step 440, the method of making a medical device includes surrounding the at least one guide member, the inner sleeve, and the support layer with a polymeric material. At step 450, the method includes forming a channel through the polymeric material. The channel passes through the first larger pick and into the at least one guide member. At step 460, the method includes extending a conductor through the channel. At step 470, the method includes electrically coupling the conductor to an electrode located above the channel.

In other embodiments, larger picks are formed over the pull wire lumens and accessed through channels using steps similar to those discussed above. In still other embodiments, larger picks are formed in areas without a guide member or pull wire lumen using steps similar to those discussed above. In these embodiments, channels are created through the larger picks to create vent or irrigation holes at desired locations along the medical device. While certain embodiments describe larger picks formed over guide members containing electrical conductors for electrodes, the disclosed braid pockets are not limited to electrical applications. In other embodiments, larger picks may be formed over pull wire lumens, fluid lumens, vent locations, or other underlying structures, or may be used to create localized openings in the braid for ventilation, fluid exchange, or access features, including in medical devices that do not include electrodes or pull wires.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A medical device, the medical device comprising:
an elongated tubular member including a proximal portion having a proximal end and a distal portion having a distal end, the elongated tubular member comprising:
an inner sleeve, the inner sleeve having an outer surface extending between an inner sleeve proximal end and an inner sleeve distal end;
at least one guide member located along a portion of the outer surface of the inner sleeve;
a braided layer surrounding the at least one guide member and the inner sleeve, the braided layer including a braid pattern extending along the at least one guide member having a consistent pick size along a first section, a consistent pick size along a second section, and a first larger pick between the first section and the second section; and
a layer of polymeric material encasing the braided layer and the at least one guide member.

2. The medical device of claim 1, wherein the braid pattern includes a consistent pick size along a third section, and a second larger pick between the second section and the third section.

3. The medical device of claim 2, further comprising a first channel extending through the first larger pick and a second channel extending through the second larger pick.

4. The medical device of claim 2, wherein the first larger pick and the second larger pick are the same size, or the first larger pick and the second larger pick are different sizes.

5. The medical device of claim 1, wherein a distal end of the at least one guide member is adjacent the inner sleeve distal end.

6. The medical device of claim 1, wherein the at least one guide member includes a first guide member and a second guide member.

7. The medical device of claim 6, wherein the first guide member is located opposite the second guide member.

8. The medical device of claim 1, further comprising at least one conductor extending through the at least one guide member.

9. The medical device of claim 1, further comprising a first electrode positioned over the first larger pick.

10. The medical device of claim 9, wherein the first electrode is electrically connected to at least one conductor extending through the at least one guide member.

11. The medical device of claim 10, wherein the at least one conductor passes through a first channel extending through the first larger pick.

12. The medical device of claim 11, further comprising a connector at the proximal end of the elongated tubular member, the at least one conductor being electrically coupled to the connector.

13. A medical device, the medical device comprising:
an elongated tubular member including a proximal portion having a proximal end and a distal portion having a distal end, the elongated tubular member comprising:
an inner sleeve, the inner sleeve having an outer surface extending between an inner sleeve proximal end and an inner sleeve distal end;
at least one guide member located along a portion of the outer surface of the inner sleeve, the at least one guide member having a first lateral opening; and
a braided layer surrounding the at least one guide member and the inner sleeve, the braided layer including a braid pattern extending along the at least one guide member having a consistent pick size along a first section, a consistent pick size along a second section, and a first larger pick between the first section and the second section;
wherein the first larger pick surrounds the first lateral opening.

14. The medical device of claim 13, further comprising a first electrode positioned over the first larger pick.

15. The medical device of claim 14, further comprising at least one conductor extending through the at least one guide member and the first lateral opening, the at least one conductor being electrically coupled to the first electrode.

16. A method of making a medical device, the method comprising:
placing at least one guide member on an outer surface of an inner sleeve;
braiding a support layer around the at least one guide member and the inner sleeve, wherein the support layer includes a braid pattern extending along the at least one guide member having a consistent pick size along a first section, a consistent pick size along a second section, and a first larger pick between the first section and the second section.

17. The method of making a medical device of claim 16, wherein the first larger pick is formed by braiding around a mandrel positioned transverse to a longitudinal axis of the support layer.

18. The method of making a medical device of claim 16, further comprising surrounding the at least one guide member, the inner sleeve, and the support layer with a polymeric material.

19. The method of making a medical device of claim 18, further comprising forming a channel through the polymeric material, the channel passing through the first larger pick and into the at least one guide member.

20. The method of making a medical device of claim 19, further comprising extending a conductor through the channel.
